(19) United States
(12) Patent Application Publication
(10) Pub. No.: US 2025/0037843 A1
(43) Pub. Date: Jan. 30, 2025

(54) SYSTEMS AND METHODS FOR MEASURING FRACTAL DYNAMICS OF DENSITY RATE VARIABILITY IN BREAST CANCER

(71) Applicant: Emanuele Raggi, Providence, RI (US)

(72) Inventor: Emanuele Raggi, Providence, RI (US)

(21) Appl. No.: 18/784,906

(22) Filed: Jul. 25, 2024

Related U.S. Application Data
(60) Provisional application No. 63/515,588, filed on Jul. 26, 2023.

(51) Int. Cl. G16H 30/40; G16H 50/20; G16H 50/70

(52) U.S. Cl. CPC G16H 30/40; G16H 50/20; G16H 50/70

(57) ABSTRACT

Systems and methods to localize and differentiate tumors from healthy tissues completely contactless exploiting the vibrational pattern analysis of optically detected surface vibrational wave mode leveraging multifractal spectral analysis. Directing pulsed wave photoacoustic excitation sources into a desired area of the body distributing acoustic energy into the tissue at the speed of sound and detecting the surface wave on the tissue may reveal specific vibrational patterns that are specific for health and cancerous tissues. These methods rely on the analysis of vibrational patterns using image post-processing techniques, numerical modeling or a digital library. The use of non-contact techniques can be used for breast cancer leading to an improved prognosis.

### CROSS-REFERENCE TO RELATED APPLICATION

This application claims the benefit of the U.S. Provisional Application No. 63/515,588 entitled, “SYSTEMS AND METHODS FOR MEASURING FRACTAL DYNAMICS OF DENSITY RATE VARIABILITY IN BREAST CANCER” filed on Jul. 26, 2023, which is incorporated herein by reference in its entirety.

### TECHNICAL FIELD

Embodiments relate generally to systems and methods to localize and differentiate tumors from healthy tissues completely contactless exploiting the vibrational pattern analysis of optically detected surface vibrational wave mode leveraging multifractal analysis and principal component analysis.

### STATEMENT REGARDING FEDERALLY SPONSORED RESEARCH

N/A

### BACKGROUND OF THE INVENTION

Up to 30% of breast cancer diagnoses are missed in the early stages (false-negative). False-negative results lead to delays in treatment and a false sense of security for affected women. Yearly breast cancer diagnoses are massive, making this an extremely serious problem. In the USA close to 288,000 women were diagnosed with breast cancer in 2022 and approximately 43,000 died due to a late diagnosis. Additionally dense breasts of younger women precludes the detection of early-stage tumors by mammography. Triple negative breast cancer (TNBC) is the most aggressive and 12% of women are yearly affected. A dense breast plays a significant role in missing a TNBC diagnosis at the early stage, decreasing the overall survival rate for the affected women. Limitations of the current mechanical transducers used in conventional sonography are mainly due to resonant devices with rather limited operating-frequency ranges and little modulation agility. Thus, there is an unmet clinical need for accurate, painless, cheap early detection methodology in the current ultrasound market.

This patent will disclose a non-contact optical surface vibrational technique analysis that is able to differentiate healthy tissue from cancerous tissues. The innovative idea is the absence of usage of gel, water or electrodes while performing an ultrasound session, saving the patient's pain due to the sonographers application of pressure on the probe, causing discomfort in the majority of cases, and eliminating the sonographer's chronic pain. A combination of two lasers working in synchronization with an external optical interferometer are able to detect skin surface waves. Given the vibrational response of the tissue to the external laser excitation, it is possible to differentiate a healthy to a cancerous tissue by understanding the surface vibrational pattern. Intensity of surface vibration results in the creation of a stiffness map, which forms the final image.

One obvious advantage of laser-based sonography is the higher comfort level to the patient due to its contact-less nature. Other advantages in laser-based sonography are due to the easy control of laser light with electronic means and laser pulses can be generated at almost any temporal pattern, thus allowing single pulses, continuous-wave operation, deep amplitude and frequency modulation. Finally, laser-based methods allow for much more agile scanning across the patient than any mechanical transducer would be capable of. In 2021 we conducted several field interviews with doctors and patients to assess the market concluding that 90% of interviewed patients would prefer not to be touched during an ultrasound session and 77% would go contactless due to the “less invasive” approach. 83% of patients would prefer more automation inside a hospital as they are aware of the danger of missed diagnoses.

The present invention relates to systems and methods for detecting and processing how the vibration on the skin of the patients is processed and analyzed can provide a terrific improvement in the patient's experience and a healthier way of localizing certain diseases given the non-contact process used.

An ultrasound wave occurs at frequencies that are above the bandwidth of human audio capabilities. In terms of imaging, the frequencies cover a wide variety of applications, including, but not limited to, underwater sonar systems, industrial non-destructive testing, near-surface damage assessment, medical diagnostics, and evaluation of acoustic micro-structures.

Regular ultrasonic imaging techniques have long-known benefits. Technological advancements in hand-held probes have been proven to provide better images for soft tissue structures such as the eyes, abdomen, brain, neck, and feet. Conventional techniques for obtaining ultrasound images typically require applying a mechanical probe directly to the patient's body, focusing on the specific area of interest for a particular period of time. The amount of time spent probing is determined by the sonographer's level of experience and by the complexity of the anomaly being investigated.

During an ultrasound, the probe emits an acoustic wave, causing a thermal dilation of tissue that consequently deforms and elasticizes the tissue, generating a returning wave. These waves ricochet back to the probe, and the time difference between the original emission from the probe and the returning acoustic wave compose the sonogram, which is typically displayed on a monitoring system.

Unlike CT scans, MRIs, or x-rays, ultrasound imaging technologies are relatively low-cost and do not involve the injection of radiation or contrast. They are also portable. Ultrasound technicians and doctors must constantly balance the quality of scans with the hospitals' needs for efficient, high volume scanning to increase healthcare reimbursements.

The use of hand held probes, however, poses problems that current technology is not yet able to overcome. The evidence is clear that overuse injuries brought on by repetitive muscle stresses associated with the performance of ultrasound exams can lead to muscular damage and, in some cases, career-ending injuries. In addition, the hand-held probe may exacerbate existing pain for patients when technicians apply pressure on the point or area under investigation. This is especially prominent during a prolonged pressure application to a painful area.

Patients may also move during the procedure due to discomfort or pain, and this can interfere with the quality of the scan, corrupt the ultrasonic reading, or elongate the duration of the exam. If patients are in extreme pain or cannot be still during a scan, the hospital may have to reschedule a session, since efficiency and patient turnover must remain high in the current profit structure.

A patient's body type and size also impact the quality of an ultrasound. With overweight and obese patients, the ultrasound wave has to pass through more fat tissue before reaching the main site, sometimes causing an unclear reading. In addition, internal gas can also interfere with the accuracy of a reading, requiring a longer session. In these cases, the sonographer must apply a great deal of pressure to the site.

In order to produce an accurate reading using current ultrasound technology, the system must always have a clear understanding of where the probe is in space. Spatial resolution is often a major obstacle with handheld devices. Accurate readings require the system to have a continuous record of the angular velocity, linear velocity, roll, pitch, and yaw. While current technology offers spherical probe transducers, which concentrate the beam, there is still too much room for human error. For example, if the technician's hand trembles or if there is inconsistency in the amount of pressure they apply, this can compromise the integrity of the reading. In addition, if the sonographer did not apply enough gel to create the ideal electrodynamic balance, the returning wave could be flawed and lead to an inaccurate scan.

In conventional sonography, a medical practitioner applies a sono-mechanical transducer to the skin or interior surfaces of a patient as close as possible to the organ of interest. The transducer both launches sound waves, and records echoes, which are caused by variations of sound-propagating properties (impedance) in the tissue. Because the sound impedance is specific for each tissue type, the echoes are correlated to physiological features. The time between launching of sound waves and reception of echoes gives the depth below the point where the transducer has been applied.

Recently, contactless sonography methods have been tested where rapid pulsed heating of the skin due to illumination with a pulsed laser generates sound waves and laser-interferometric methods measure the vibration of the skin due to returning echo. One obvious advantage of laser-based sonography is the higher comfort level to the patient due to its contact-less nature. There are, however, other less immediately obvious advantages in laser-based sonography due to the easy control of laser light with electronic means: Whereas the mechanical transducers used in conventional sonography are resonant devices with rather limited operating-frequency ranges and little modulation agility, laser pulses can be generated at almost any temporal pattern, thus allowing single pulses, continuous-wave operation, deep amplitude and frequency modulation, and so on. This capability allows the implementation of sophisticated sensing techniques that are based on recognizing and categorizing vibrational patterns.

Finally, laser-based methods and optical vibrational-based methods allow for much more agile scanning-detection across the patient than any mechanical transducer would be capable of. To our knowledge, current work is not yet exploiting the advantages of surface vibrational pattern differentiation on human skin.

Research and experimentation into pulse or continuous lasers to be used as contactless ultrasonic devices is in the initial stages. Some studies have proven that contactless ultrasound techniques can be a useful tool for both humans and animals. Eye-safe techniques are already being used to treat optical problems such as myopia and are considered safe at a particular exposure. Wireless portable ultrasound probes are already in use and present a great advantage in terms of portability and field operations. They do not, however, eliminate the need to physically touch the patient nor do they limit sonographers' stress injuries. This demonstrates a need for developing novel techniques and processes in this field that allow for acoustic waves to enter soft tissue, skin, and organs without the need for physical contact with the patient and based on the vibration that is received on the surface of the skin, precisely categorize that vibration.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later. In order to overcome the aforementioned limitations in current detection technology, the present invention utilizes non-contact optical vibration-based techniques on the skin tissue to reveal specific vibrational patterns that are specific for healthy and cancerous tissues. These methods rely on the combined analysis of mechanical vibrational patterns, multifractal analysis of the spectra of the vibration. On examination, breast cancer might be deforming the breast tissue giving a distorted or enlarged shape, and the skin appears like the skin of an orange (Peau d'orange), warm and thick to touch. In other occasions nipple is seen as crusting, flattening, blisters, or even retracting changing its original shape. A discrete palpable mass may or may not be present. However, these changes in appearance but also in density or color can be analyzed specifically from understanding the vibrational pattern. After building a specific vibrational database of data composed of healthy tissues and cancerous tissues, it will be possible to create a library and compare the current sample with existing collected images to understand the multifractal spectral analysis.

The analysis of vibrational patterns on the skin of the patient following non-contact laser excitation techniques can be used for differentiation between healthy or diseased tissues.

### BRIEF DESCRIPTION OF THE DRAWINGS

The novel features that are characteristic of the present invention are set forth in the appended claims. However, the invention's preferred embodiments, together with further objects and attendant advantages, will be best understood by reference to the following detailed description taken in connection with the accompanying Figures:

FIG. 1 is an image obtained from a laser ultrasonic technique where the ring shape represents the maximum vibrational intensity detected on the surface of a phantom material mimicking the human skin. The red box on the left of the image will be used through this patent to provide an example of vibrational technique differentiation. In this portion of the image the healthy tissue is analyzed.

FIG. 2 is an image taken from the red box area mentioned on FIG. 1.

FIG. 3 is an image showing the difference in multifractal spectrum in various zones of the image (specifically, it points out the healthy tissue).

FIG. 4 is an image obtained from a laser ultrasonic technique where the ring shape represents the maximum vibrational intensity detected on the surface of a phantom material mimicking the human skin. The red box on the left of the image will be used through this patent to provide an example of vibrational technique differentiation. In this portion of the image the cancerous tissue is analyzed.

FIG. 5 is an image showing the difference in multifractal spectrum in various zones of the image (specifically, it points out the tumor tissue area with an arrow).

FIG. 6 is an image obtained from a laser ultrasonic technique where the ring shape represents the maximum vibrational intensity detected on the surface of a phantom material mimicking the human skin. The red box on the left of the image will be used through this patent to provide an example of vibrational technique differentiation. In this portion of the image the middle area of the tumor and healthy tissue is analyzed.

FIG. 7 is an image showing the difference in multifractal spectrum in various zones of the image (specifically, it points out the middle area of the tumor tissue with an arrow).

FIG. 8 is an image showing the comparison of the various multifractal spectra with the different types of tissues.

FIG. 9 is an image showing how the energy of the image is distributed. In this case it is the sum of squared elements in the normalized histogram.

### DETAILED DESCRIPTION OF THE INVENTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

The ability to describe signal regularity is important when dealing with phenomena that have no characteristic scale at first sight. The applications of different techniques to analyze a historical data series or, more generally speaking, a set of data, can make the difference when properly interpreting the data and avoiding making incorrect conclusions or wrong assumptions leading to false misinterpretations. When data can't be interpreted properly or the stochasticity keeps the analyst from drawing a meaningful conclusion, a useful tool that can be used to make sense and properly steer the data set is the power spectral density, known also as PSD.

Power spectral density (PSD) estimation comes handy in case of translationally invariant data series, meaning that routine measures such as mean and variance do not change over time. Instead, data series that seem to lack characteristic scale are considered scale-invariant. For example, if we manipulate a data series changing the time axis (typically intended as x axis on a cartesian coordinate reference frame) the result does not change: in fact it remains unchanged or invariant to change. Scale-invariant is in fact the biggest obstacle of signal processing. Not offering any clear way of understanding how a data series behaves poses a serious problem in utilizing classical signal processing theory and its related features. Therefore, based on those limitations, the branch of fractal analysis techniques can provide unique insights on the data series being analyzed. Non-linear power laws are the most complicated series to understand because while some signals might show a power-law PSD, other signals are mainly characterized by noise. Noise, therefore, plays a pivotal role within a power-law PSD usually entailing corruption of the signal, or inability to properly discern the signal from the noise. As a way of example, a typical power law in signal analysis has a PSD of the form C|ω|^{-α} for some positive constant C and some exponent α. In order to make a proper interpretation of the signal, estimating the exponent α is going to be very important. If interpreted properly, the power law might support a conclusion. Otherwise, if interpreted poorly, may lead to the wrong assumptions. The most basic example to understand this mechanism can be offered by analyzing brownian processes and brown noise, which in fact is a type of nonlinear power law.

In the power law equation introduced before (C|ω|^{-α}), a brownian noise is characterized by having a positive exponent of α=2. Therefore the previous equation can be rewritten as C|ω|⁻².

Interpreting results from data series can be challenging and time-consuming, especially if the amount of data is significant. Multifractal analysis provides a solid way to describe real-world signals that exhibit nonlinear power-law behavior and that depend on higher-order moments and scale. In signal theory there are four types of processes that are considered the most important and those are: monofractal, multifractal, persistent or antipersistent. Knowing if a process is monofractal, multifractal, persistent or antipersistent can equip the analyst with important information about analyzing a data series and, more importantly, decide if the result makes sense or not.

Monofractals processes are defined as those processes that are homogeneous and characterized by having the same scaling properties, while multifractal processes are defined as having a nonlinear law. The brownian noise mentioned before is therefore an example of a monofractal process.

As an additional example, imagine a researcher needs to analyze two time series T1 and T2 with a certain number of samples for each series. T1 and T2 have the same amount of samples. From an initial analysis, the data do not reveal any specific information, no peaks, no valleys or strange behaviors, PSD estimates and autocorrelation sequences are also very similar too and. T1 and T2 seem to have very similar statistics and even when calculating variances, the values are almost identical. Therefore no statistical meaningful conclusions can be drawn from those two data series. The analyst may therefore be wrongly led to assign no significance to those data and even pass to another dataset.

The researcher now applies a different technique called fractal analysis. The fact that these signals are very different is now evident. After compute and plot the multifractal spectra of the two signals it is now clear that the time series are different and the behavior is different. Therefore, the researcher can conclude that the multifractal spectrum effectively shows the distribution of scaling exponents for a signal, and that the multifractal spectrum provides a measure of how much the local regularity of a signal varies in time. Wavelet leader techniques are the most robust. It is well known that the use of leaders instead of wavelet coefficients brings significant improvements in multifractal estimation and interpretation of the data. A multifractal signal shows variations in signal regularity over time contrary to a monofractal signal.

Additionally, antipersistent processes are characterized by short memory while persistent processes are characterized by long memory. Whether a process is antipersistent or persistent is useful in predicting the future behavior of a data series, and therefore the outcome of a result; also changing initial parameters and making comparisons with the initial data can be useful in understanding differences in future treatment. In the case of this patent, varying parameters in the post-processing phase can help understanding the situation of a cancerous tissue, whether or not a drug is working or simply as a monitoring solution. A data time series with long memory which is increasing over time can be expected to continue this trend. While a data time series that showed antipersistence can be expected to go the opposite way and therefore descend. For example, the diffusion of a drug through the body can portray the difference of how well the drug is taken from the patients and the analysis of multifractal spectrum can indicate a lower global regularity when compared to drug responders. This can be directly related to the increase or shrinkage of the tumor in the tissue and the analysis of surface vibration following external excitation can differentiate the two populations of respondents vs non-respondents. Lastly, persistent time series have the tendency to go back to its mean value.

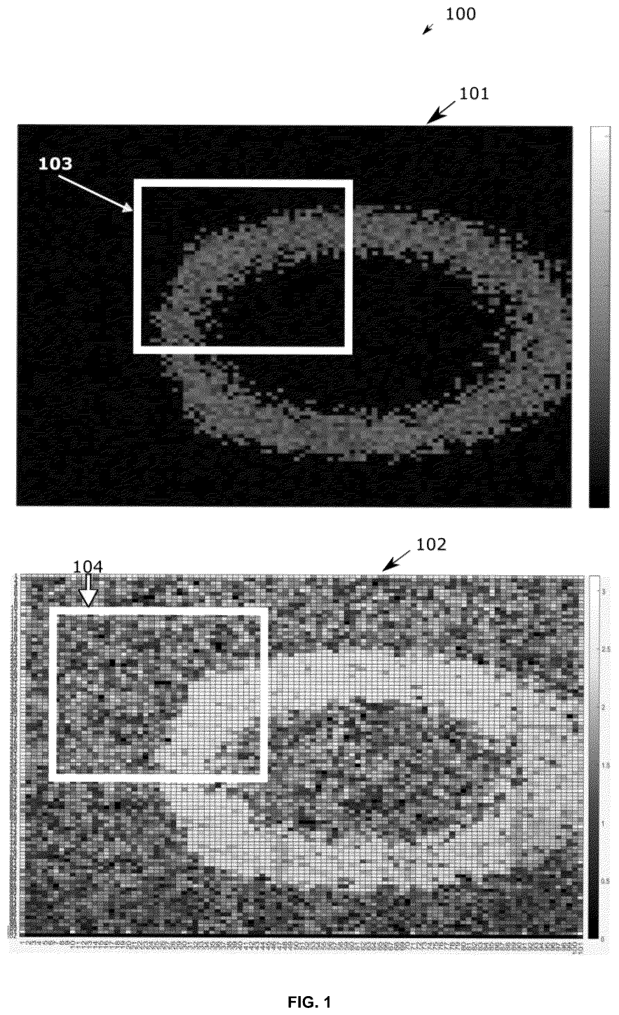

In this work the main idea is that a laser vibration optically detected on the surface of the skin is characterized by a different vibrational mode depending if the laser beam hits a healthy tissue or a cancerous tissue within the body. We can think of a time series distribution of a healthy tissue as the times series T1 and the time series of a cancerous tissue as the series T2. From T1 to T2 there is a change in density. This change in density can be demonstrated by applying the multifractal analysis in the different time series. Further, reductions in this nonlinear scaling behavior are good predictors of diseased and non-diseased tissues. As an example of an induced change in the fractal dynamics of breast density rate variability, consider the image in FIG. 1. The image in FIG. 1 is an image obtained from a laser ultrasonic technique where the ring shape represents the maximum vibrational intensity detected on the surface of a phantom material mimicking the human skin. The red box 103 from image 101 on the left of the image will be used through this patent to provide an example of vibrational technique differentiation. The image 102 is a representation of entropy values extracted from the image 101. Each and every pixel of the image 102 is color-coded. The color coding is a representation of the intensity of the vibration detected on the surface of the phantom material. Throughout this patent we will use the red box 103 as a reference example image (and its equivalent 104) as a way to differentiate healthy and cancerous tissues.

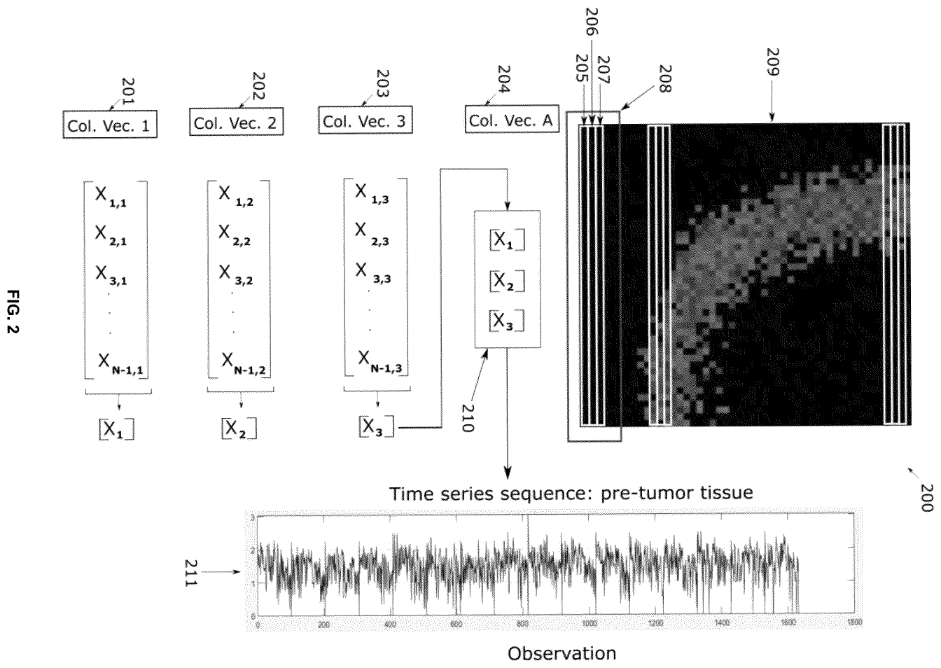

At this point we analyze the data in FIG. 2, specifically the image 209. As explained before, this image is obtained following a laser ultrasonic session on a specific area which is subjected to suspicious presence of cancer. In order to characterize and properly divide the tissues we operate a distinction in the image 209. As an example the columns 205, 206 and 207 are representing columns with healthy tissue. Specifically we are considering the columns within the box 208. We can represent each one of those columns as a column vector, where each index is the position of each pixel contained within that column. Therefore, 201 is the representation of the column vector of values of 205. 202 is the representation of the column vector of values of 206 and lastly, 203 is the representation of the column vector of values of 207. After we extracted those values, which are for convenience expressed as [X₁], [X₂] and [X₃], we are able to combine them into a unique column vector as shown in 210. This will give us the possibility to finally plot the data series related to the area of healthy tissue which is shown on 211. We will call this time series, series T1. T1 is known as the pre-tumor time series or healthy time series.

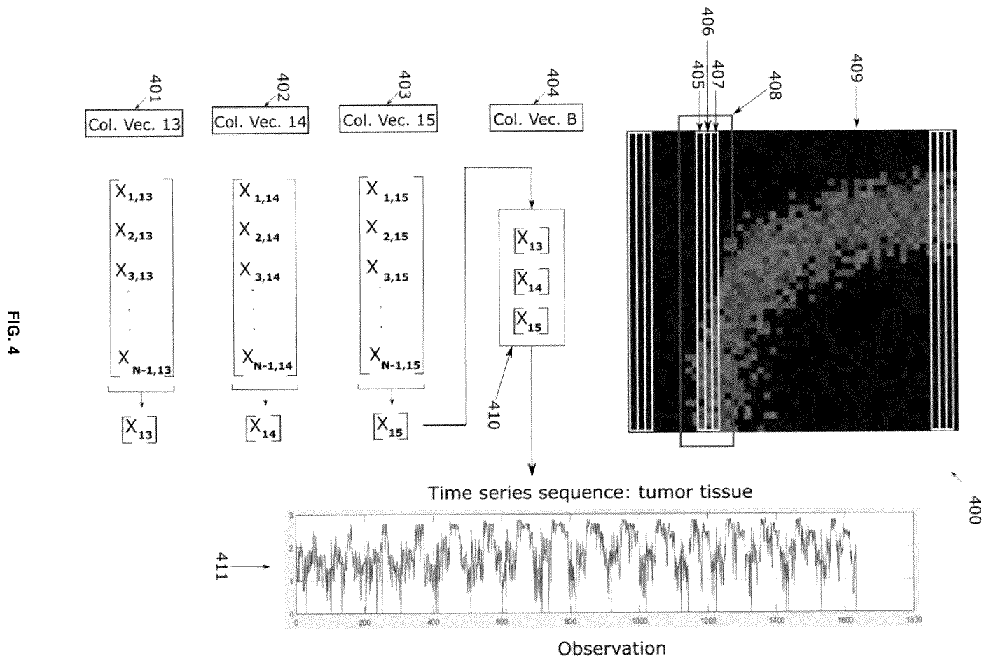

Following the creation of the first time series we can proceed to the next time series, which is the tumor time series. At this point we analyze the data in FIG. 4, specifically the image 409. As explained before, this image is obtained following a laser ultrasonic session on a specific area which is subjected to suspicious presence of cancer. In order to characterize and properly divide the tissues we operate a distinction in the image 409. As an example the columns 405, 406 and 407 are representing columns with cancerous tissue. Specifically we are considering the columns within the box 408. We can represent each one of those columns as a column vector, where each index is the position of each pixel contained within that column. Therefore, 401 is the representation of the column vector of values of 405. 402 is the representation of the column vector of values of 406 and lastly, 403 is the representation of the column vector of values of 407. After we extracted those values, which are for convenience expressed as [X₁₃], [X₁₄] and [X₁₅], we are able to combine them into a unique column vector as shown in 410. This will give us the possibility to finally plot the data series related to the area of cancerous tissue which is shown on 411. We will call this time series, series T2. T2 is known as the tumor time series.

We can see how on a visual examination of the time series depicted on 211 and 411 they seem to have an unclear trend and behavior. Therefore if we were to analyze simply the time series, there is nothing we could say about those data sets.

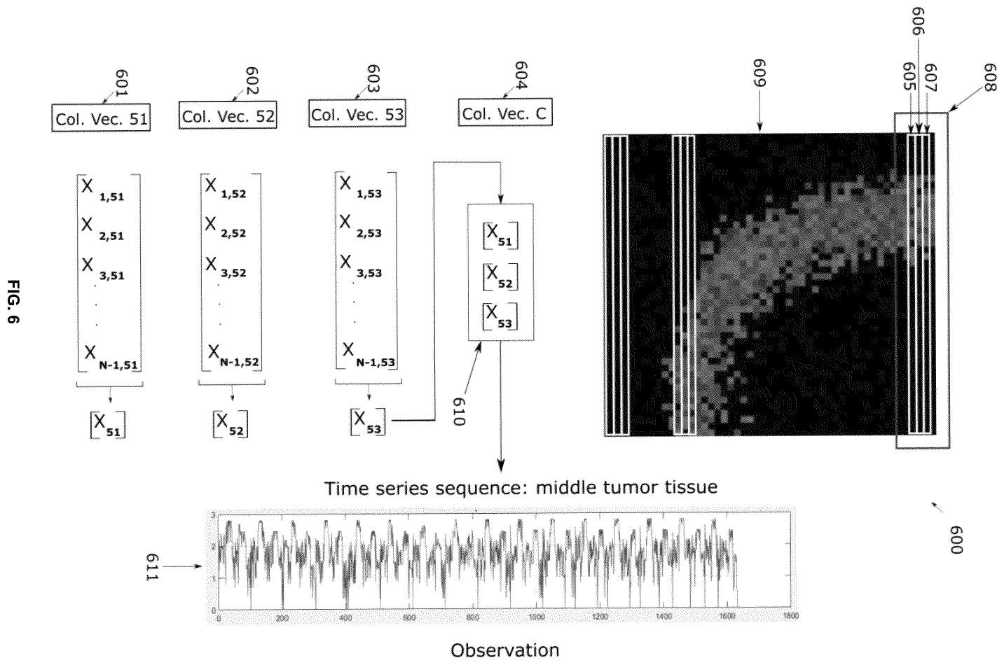

Lastly, following the creation of the second time series we can proceed to the next time series, which is the middle tumor time series. At this point we analyze the data in FIG. 6, specifically the image 609. As explained before, this image is obtained following a laser ultrasonic session on a specific area which is subjected to suspicious presence of cancer. In order to characterize and properly divide the tissues we operate a distinction in the image 609. As an example the columns 605, 606 and 607 are representing columns with different types of tissues. Those can be visualized from seeing the intensity of the vibration which is color-coded. The gray area is the pixel with intensity and the black area is the pixel with low intensity. We are considering, for example, the columns within the box 608. We can represent each one of those columns as a column vector, where each index is the position of each pixel contained within that column. Therefore, 601 is the representation of the column vector of values of 605. 602 is the representation of the column vector of values of 606 and lastly, 603 is the representation of the column vector of values of 607. After we extracted those values, which are for convenience expressed as [X₅₁], [X₅₂] and [X₅₃], we are able to combine them into a unique column vector as shown in 610. This will give us the possibility to finally plot the data series related to the area of cancerous tissue which is shown on 611. We will call this time series, series T3. T3 is known as the middle tumor time series.

We can see how on a visual examination of the time series depicted on 211, 411 and 611 they seem to have an unclear trend and behavior. For time series T2 and T3 it is impossible to differentiate anything. Therefore if we were to analyze simply the time series, there is nothing we could say about those data sets.

The data of the three time series T1, T2 and T3 do not reveal any specific information, no peaks, no valleys or strange behaviors, PSD estimates and autocorrelation sequences are also very similar too and T1, T2 and T3 seem to have very similar statistics and even when calculating variances, the values are almost identical. Therefore no statistical meaningful conclusions can be drawn from those three data series.

Therefore, the next step we apply is the multifractal spectrum analysis of the time series using the wavelet leader. We can see how the analysis of the data series of the surface vibration on the skin of the patient can be precisely differentiated.

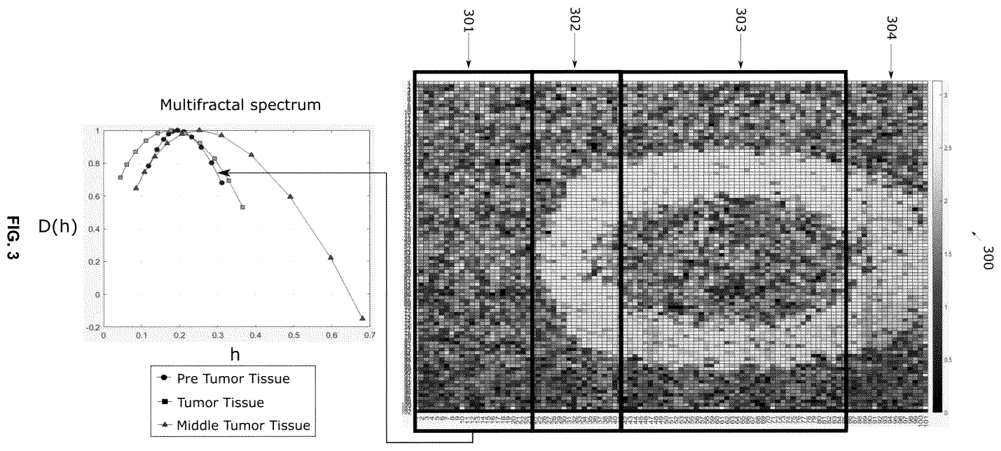

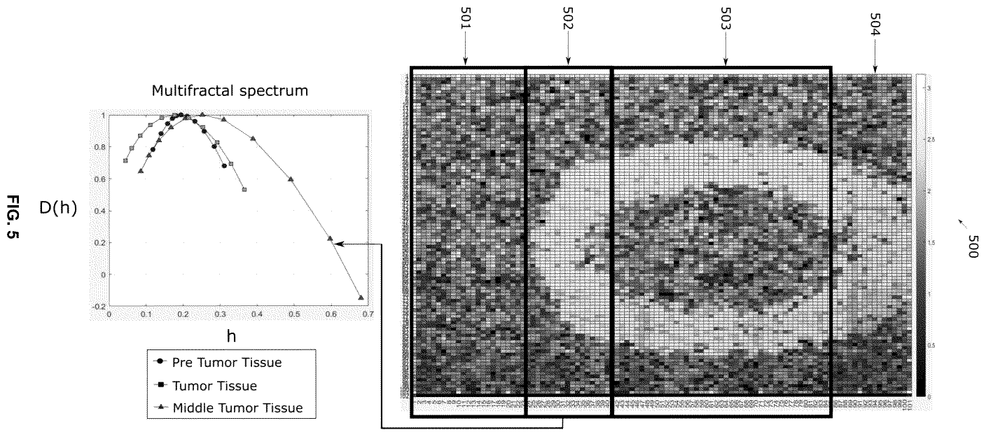

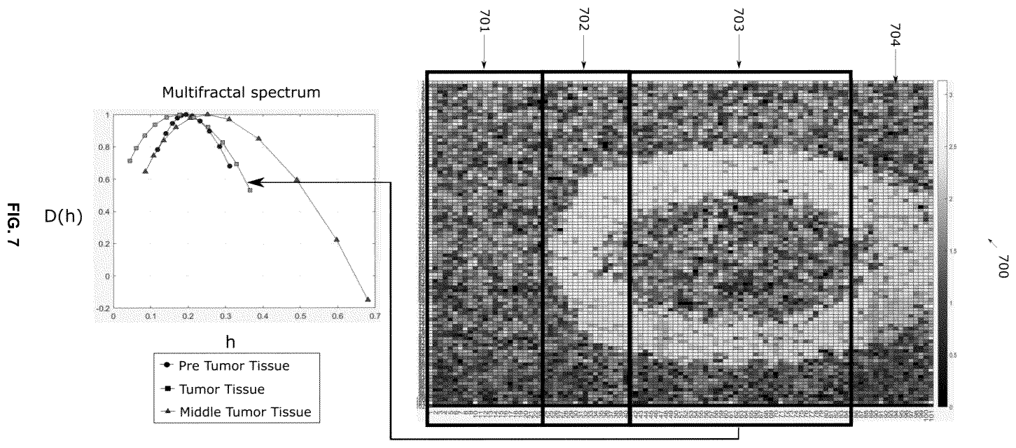

Following the calculation of the time series T1 (pre-tumor tissue), the time series T2 (tumor tissue) and the time series T3 (middle tumor) we can appreciate how the multifractal techniques can discriminate between the time series. All the time series have been processed using the discrete wavelet leader techniques also called dwtleader (discrete wavelet transform leader). FIG. 3, FIG. 5 and FIG. 7 show significant differences among time series when calculating the multifractal spectrum.

We can see how the area that is characterized by a change in density (i.e. the transition between pre-tumor tissue and tumor tissue) has a very different multifractal spectrum. The result of density change is also the result of physiologic variation of the body due to the presence of cancer. The areas that are typically affected by cancer are characterized by a change in thermal capacitance of the tissue, heat increase, change in density and blood flow values.

We expected to see a decrease in the width of the fractal spectrum between the two series (pre-tumor tissue and tumor tissue). This in fact was confirmed after running the experimental analysis. This indicates a significant decrease in the nonlinear dynamics of the breast density as measured by rate variability. In this case, the decrease of the fractal dimension in the pre-tumor tissue was the proof of density change.

We can see how the area that is characterized by a change in density (i.e. the transition tumor tissue and middle tumor tissue) has also a very different multifractal spectrum. This also communicates that this technique can differentiate the multifractal spectrum between sunset tumor tissue and an area where there is variability within the tumor itself. Specifically, the middle tumor area is composed of sub-areas where there is healthy tissue and tumor tissue. This means that variability can be detected on multiple levels.

Clearly we applied this technique also to differentiate the pre-tumor area and the middle tumor giving multifractal spectra different from each other.

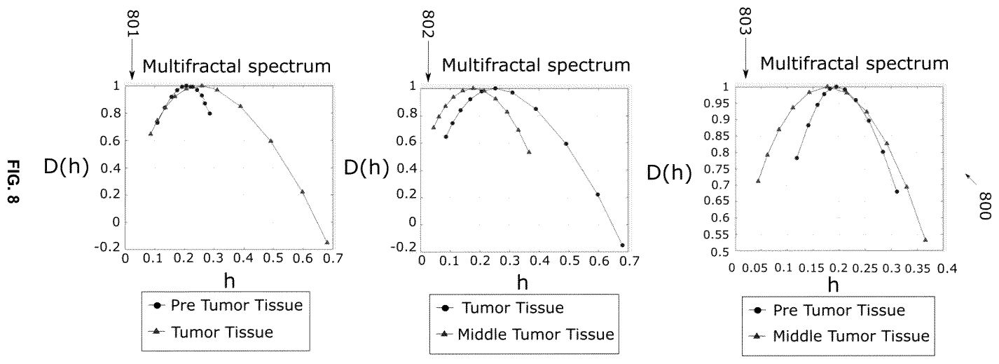

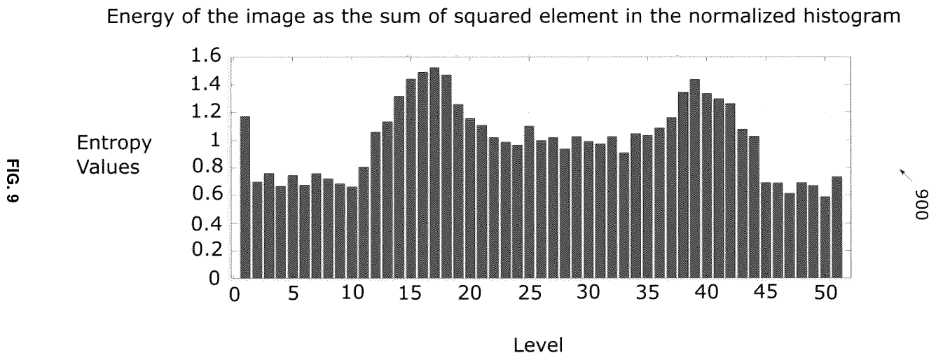

Finally on FIG. 8 we depicted all the possible cases as shown on 801, 802 and 803. Multifractal spectrum of the tumorous area is greater than non-tumors area, therefore time series characterized by healthy tissue will tend to cluster similar to 801, whereas different tumorous areas will tend to cluster as 802 or 803. Finally in FIG. 9 it is shown the energy distribution of the image 101. This histogram is showing how the energy of the image is distributed. In this case it is the sum of squared elements in the normalized histogram. The higher energy bins are obtained at the borders resembling a ring shaped image. This confirms that after applying multifractal analysis the histogram of the energy distribution of the image can also be used as a marker of the disease following the vibrational pattern.

Significant increase in the width of the multifractal spectrum is a marker of density dysfunction. So, specific vibrations detected in the tumor area can be used as a non-invasive biomarker for detection of breast cancer.

The present patent can also be used to point out differences in Triple Negative Breast Cancer (TNBC), and Inflammatory Breast Cancer (IBC) which very often doesn't appear as a lump in the breast but rather as a density change in some areas of the breast. Therefore, the present technique can be used to diagnose those patients in a non-invasive way, offering a viable alternative to the current standard of care which makes use of invasive devices or biopsies in other cases. This is also important in the case of patients with TNBC given the fact of the high lethality of the disease. Therefore, for TNBC, we expect to see significant increase in the width of the multifractal spectrum in TNBC phantom material and use that as a marker of density dysfunction.

The methodology disclosed in this patent can be applied both for real-time measurements and for post-processing measurements. Based on the clustering of the similar vibrational pattern it would be possible to assess the progress or the shrinkage of a disease or the effectiveness of a drug. Lastly, based on the clustering information, this technique can be used to predict potential responders and non-responder for chemotherapy treatment and potentially locate ahead of time the non-responders in order to assess alternative solutions.

### LIST OF EMBODIMENTS AND EXAMPLES

Specific systems and methods of locating cancerous to non-cancerous tissues have been described. The detailed description in this specification is illustrative and not restrictive or exhaustive. The detailed description is not intended to limit the disclosure to the precise form disclosed. Other modifications besides those already described here are possible without diverting from the inventive concepts described in this specification, as will be clear to those skilled in the art. When the specification or claims recite methods, steps or functions in a specific order, alternative embodiments or modifications may perform the tasks in a different order or can be substantially concurrent. However, the inventive subject matter is not to be restricted except in the spirit of the disclosure listed in this specification.

During the interpretation of the disclosure, all terms should be interpreted in the broadest possible manner consistent with the context and the terms described. Unless otherwise defined, all technical and scientific terms used in this specification have the same meaning as commonly understood by one of ordinary skills in the art to which this invention belongs. The terminology used in this specification is not intended to limit the scope of the invention, which is defined solely by the claims. This invention is not limited to the particular methodology, systems described in this specification and, as such, can vary in practice.

What is claimed is:

1. A method for differentiating cancerous tissue from healthy tissue of a subject using a multifractal analysis in real-time comprising the steps of:
a) obtaining a laser image following a laser ultrasonic session on a specific area of the breast;
b) assigning values of vibrational intensity to each pixel in the image;
c) color-coding the vibrational intensity for each pixel in the image;
d) dividing the image in different column vectors where every value within the column vector is the pixel intensity originating from the vibration at that point;
e) clustering the column vector according to vibrational similarity in order to create areas of similar vibrational pattern;
f) combining all the column vectors for the specific group into a single and unique column vector;
g) applying the discrete wavelet transform leader to calculate the multifractal spectrum for each group;
h) plotting the different multifractal spectrum for each group; and
i) identifying healthy tissue from cancerous tissues.

2. The method of claim 1, wherein the image can be obtained from any part of the body.

3. The method of claim 1, wherein the multifractal spectrum can be used for monitoring the status of a disease.

4. The method of claim 1, wherein the multifractal spectrum can be used for monitoring the effectiveness of a drug of shrinking a tumor.

5. A method for differentiating cancerous tissue from healthy tissue of a subject using a multifractal analysis in post-processing comprising the steps of:
a) loading an image obtained from a previous laser ultrasound session;
b) assigning values of vibrational intensity to each pixel in the image;
c) color-coding the vibrational intensity for each pixel in the image;
d) dividing the image in different column vectors where every value within the column vector is the pixel intensity originating from the vibration at that point;
e) grouping the column vector according to vibrational similarity in order to create areas of similar vibrational pattern;
f) clustering all the column vectors for the specific group into a single and unique column vector;
g) applying the discrete wavelet transform leader to calculate the multifractal spectrum for each group;
h) plotting the different multifractal spectrum for each group; and
i) identifying healthy tissue from cancerous tissues.

6. The method of claim 5, wherein the multifractal spectrum can be used for monitoring the future status of a disease.

7. The method of claim 5, wherein the multifractal spectrum can be used for predicting the status of a disease.

8. The method of claim 5, wherein the multifractal spectrum can be used for predicting responders and non-responder for chemotherapy treatment.
